(19) United States
(12) Patent Application Publication
(10) Pub. No.: US 2004/0191783 A1
(43) Pub. Date: Sep. 30, 2004

(54) Low density micro-array analysis in human breast cancer

(76) Inventors: Guy Leclercq, Bruxelles (BE); Jose Remacle, Malonne (BE); Marc Lacroix, Baelen (BE); Nathalie Zammatteo, Gelbressee (BE); Francoise de Longueville, Natoye (BE)

Correspondence Address: Bell, Boyd & Lloyd, LLC, P.O. Box 1135, Chicago, IL, 60690-1135, US

(21) Appl. No.: 10/404,743

(22) Filed: Mar. 31, 2003

(51) Int. Cl. C12Q 1/68; C12M 1/34

(52) U.S. Cl. 435/6; 435/287.2

(57) ABSTRACT

A method and kit comprising reagents for detection and/or quantification of polynucleotide or polypeptide sequences potentially present in a sample, said sequences representing the gene expression associated with different cell phenotypes and functions and differentiating the gene expressed in cancer tissue compared to normal or reference material. The method and kit are especially suited for the identification and/or characterization of cancer tissues and for follow up of patient treatments.

### BACKGROUND OF THE INVENTION

The present invention pertains to the field of diagnosis and prognosis of human breast cancer. In particular, the present invention pertains to a method of diagnosing the onset of breast cancer and allowing a reliable prognosis of its future development. In addition, the present invention relates to a micro-array, containing selected polynucleotides or polypeptides, which enable the quantification of particular differentially expressed genes in tumors for a precise diagnosis and prognosis and eventually curative follow up of breast tumor patients.

In Western Countries about 1 out of 11 women develop breast cancer, which is second only to lung cancer in tumor associated diseases. Breast cancer is a very heterogeneous disease with a number of so far recognized and still unknown factors being involved. Female hormones have been found to exhibit a significant impact on oncogenes, the transcription and overexpression, respectively, of which may result in the development of breast cancer, including e.g. the amplification of HER-2 and the epidermal growth factor receptor genes, and overexpression of cyclin D1. Likewise, genetic alterations or the loss of tumor suppressor genes, e.g. p53, have been found to also account for the occurrence of breast cancer. Also, in the recent past, two genes termed BRCA1 and BRCA2 have been characterized which are supposed to be implicated in pre-menopausal familial breast cancer.

A number of factors are deemed to increase a woman's risk of having the disease, including age, history of prior breast cancer, exposure to radiation, hereditary history, upper socioeconomic class, nulliparity, early menarche, late menopause, or age at first pregnancy greater than 30 years. Also, prolonged use of oral contraceptives and long-lasting postmenopausal estrogen replacement are considered to add to the risk.

Despite the considerable progress in the molecular understanding of the various causatives of breast cancer as well as progresses made in the treatment thereof, e.g. in radio-, chemo-, and hormone-therapy, more than one-third of female patients still succumb to the disease. In most cases death results from a dissemination of cancer cells and their proliferation at secondary sites.

It is well acknowledged in the art that a diagnosis of breast cancer as early as possible is vital to secure a most favorable outcome for treatment. For this reason, many countries with advanced healthcare systems have instituted screening programs for breast cancer, such as mammography. Abnormal tissues detected during these screening procedures are typically investigated in more detail by clinico-pathological analysis methods. However, due to the cumbersome and sometimes long lasting experimentation involved, new approaches are needed to ensure a better characterization and treatment of the extensively heterogeneous breast tumors.

One major problem of a generalized use of new technologies in oncological routine is their complexity, their low prognostic relevance due to different factors being involved and above all the costs involved.

In the past few decades, molecular biology techniques have raised some hope to replace conventional clinico-pathological techniques. To this end a tissue sample, suspected to have developed cancer, is investigated primarily on the basis of genes, in particular their expression, which genes are known and/or suspected to be involved in the onset and progression of cancer.

So far a variety of different genes have been characterized and found or suspected to be involved in different subtypes of breast cancer.

In WO/0210436 a particular set of genes is disclosed that are differentially expressed in tumors characterized as high or low mitotic index activity (MAI) tumors.

In WO/0175160 a method for the stratification of a cancer patient population into various cancer therapy groups is disclosed, based on an analysis by genomic DNA micro-array of multiple gene amplifications or deletions present or absent in the abnormal tissue of each patient. In particular, the teaching laid down herein involves patient stratification into one of at least four cancer therapy groups based on the micro-array analysis of gene amplification or gene deletion at multiple chromosome locations.

WO/055173 refers to a list of polynucleotides and polypeptides for the detection, prevention and treatment of disorders in the female reproductive system, particularly breast cancer. The document illustrates potential sequences of interest which are supposed to be related to breast or ovarian tumors but does not give any classification of cancers.

In WO/9906831 there are disclosed genes associated with the development of estrogen independent malignant cell growth, i.e. the BCAR1, BCAR2 and BCAR3 genes.

Both of WO/02059271 and WO/0194629 propose a list of genes found to be differentially expressed in normal tissue and breast carcinomas. The only gene classification presented in these documents is based on differences between infiltrating lobular and ductal carcinomas, which relates to the sublocalization of cancer in the mammary gland.

WO/0151628 refers to polynucleotide sequences which were identified through subtracted library to be differentially expressed in breast carcinoma. This publication proposes a classification of the genes according to the origin site of cancer: invasive lobular carcinomas (ILC), clinical invasive ductal carcinomas (IDC) and clinical ductal carcinomas in situ (DCIS) versus normal breast tissue samples. Genes are also classified according to the aggressiveness of the tumor.

All of the above mentioned documents based their findings on a different expression of particular genes in either normal tissue and tumor tissue, concluding that the differentially expressed genes should be involved in the onset and/or progress of cancer.

In principle, gene expression may be determined both at the transcriptional (mRNA) and at the translational (protein) level. Methods on the basis of proteins are presently still difficult to use and are limited in the number of proteins to be detected simultaneously. For this reason the main focus resides on assays for gene expression in a biological sample by qualitative and quantitative analysis of its mRNA population (transcriptome), which may be carried out through the use of the so-called “DNA micro-arrays” or “DNA biochips”.

These DNA micro-arrays comprise solid surfaces bearing multiple cDNA- or oligo- or polynucleotides spotted thereon, that play the role of so called capture probes. These capture probes, which represent genes or parts of genes of different length, e.g. between 10 and 1500 nucleotides, are either chemically synthesized in situ on the surface or laid down using a special device, the “arrayer” (cDNA-based arrays).

In most studies involving micro-arrays, labeled target cDNAs obtained by reverse transcription from the population of cellular mRNAs are incubated with the array, and the amount of material hybridized to the specific capture probes is determined by various techniques, such as e.g. radioactivity, colorimetry or fluorescence. Micro-arrays have the inherent advantage to detect the expression of genes in parallel with a direct read out of the hybridization results.

However, most of the DNA micro-arrays commercially available carry several thousands of capture probes and are, therefore, due to this vast amount of sequences to be synthesized, purified, quantified, and to be fixed on the solid support quite expensive and require a rather complicated data analysis. Moreover, they may carry many capture probes devoid of real interest in a perspective of routine breast cancer study, because these are specific to genes unexpressed, unvariable, or which expression level has never been explored in that kind of cancer. Thus, although these “high-density” DNA micro-arrays may give to the basic researcher a means to identify potential novel mRNAs regulated in different tissues at different stages of their normal or abnormal development, they do not provide a data/price ratio high enough to satisfy the clinician's desire for a tool applicable for routine analysis in their everyday clinical activities.

Accordingly, there remains a need in the art for means that permits a more accurate diagnostics, prognosis and therapy follow up of breast cancer.

### SUMMARY OF THE INVENTION

In consequence, a problem of the present invention resides in providing a novel means for allowing a rapid and reliable diagnosis and prognosis of breast cancer, which may be produced at low costs and which may be performed rapidly and without difficulty.

This problem has been solved by providing a micro-array for the diagnosis and prognosis of human breast cancer that contains a solid support onto which a plurality of particular polynucleotides or polypeptides are present in the form of an array. These polynucleotides/polypeptides (or fragments thereof) are selected such that specific genes or their complementary, or their products (or fragments thereof) are represented thereon essentially corresponding to two major categories, namely (A) at least one gene or a fragment thereof representing at least 4 out of 6 phenotypes, preferably 5 out of 6 phenotypes, more preferably 6 out of 6 phenotypes, selected from luminal/epithelial, basal/myoepithelial, mesenchymal, the ErbB2, the hormonal phenotypes and the hereditary susceptibility to breast cancer, and (B) at least 10 genes associated with at least 3 cellular and/or house keeping functions.

The above categories A and B are illustrated in more detail in table I below:

TABLE I
Examples of gene groups related to the categories A and B
A) Genes related to a breast cancer phenotype

| | |
|---|---|
| 1 | Luminal/Epithelial phenotype |
| 2 | Basal/myoepithelial phenotype |
| 3 | Mesenchymal phenotype |
| 4 | ErbB2 phenotype |
| 5 | Hormonal phenotype |
| 6 | Hereditary phenotype |

B1) Cellular Gene Functions

| | |
|---|---|
| 1 | Adhesion |
| 2 | Cell cycle regulation and proliferation |
| 3 | Chemoresistance |
| 4 | Angiogenesis |
| 5 | Protein processing and turnover including cleavage, synthesis, |
| | stabilization and transport, proteolysis |
| 6 | Oxidative metabolism |
| 7 | Inflammatory response |
| 8 | Cell structure |

B2) House keeping genes

Table II illustrates some specific examples for all of the categories A and B genes:

TABLE II
List of 210 genes whose mRNA level is indicative of breast cancer tissues or cells

| | | A | |
|---|---|---|---|
| | | BREAST CANCER | B |
| GENE NAME | GENE PRODUCT NAME(S) | PHENOTYPE | CELL FUNCTION |
| ABCB1 | ATP-binding cassette, sub-family B, member 1; P- | | Chemoresistance |
| | glycoprotein; Multidrug resistance protein 1 (MDR1) | | |
| ABCC1 | ATP-binding cassette, sub-family C, member 1; | | Chemoresistance |
| | Multidrug resistance-associated protein (MRP, MRP- | | |
| | 1) | | |
| ABCG2 | ATP-binding cassette, sub-family G, member 2; | | Chemoresistance |
| | Breast cancer resistance protein (BCRP); | | |
| | Placenta-specific ATP-binding cassette transporter | | |
| | (ABCP) | | |
| ACTR1A | ARP1 actin-related protein 1 homolog A | Hereditary phenotype | Cell structure |
| AIB3 | Thyroid hormone receptor binding protein (TRBP); | Hormonal phenotype | |
| | Cancer-amplified transcriptional coactivator ASC-2; | | |
| | Nuclear receptor coactivator RAP250; Peroxisome | | |
| | proliferator-activated receptor interacting protein | | |
| | (PRIP); KIAA0181 protein | | |
| APEX | APEX nuclease (multifunctional DNA repair enzyme) 1 | Hereditary phenotype | |
| ARVCF | Armadillo repeat gene deletes in velocardiofacial | Hereditary phenotype | Adhesion |
| | syndrome | | |
| ATM | Ataxia telangiectasia mutated | | Cell cycle regulation and |
| | | | proliferation |
| BAG1 | BCL-2-associated athanogene | | Cell cycle regulation and |
| | | | proliferation |
| BAK1 | Bcl-2-antagonist/killer 1 | | Cell cycle regulation and |
| | | | proliferation |
| BAX | BCL2-associated X protein | | Cell cycle regulation and |
| | | | proliferation |
| BCAR1 | Breast cancer anti-estrogen resistance-1; | Hormonal phenotype | |
| | p130Cas adaptor protein | | |
| BCL2 | B-cell CLL/lymphoma 2 | | Cell cycle regulation and |
| | | | proliferation |
| BCL2L1 | Bcl-2-like 1; Bcl-X | | Cell cycle regulation and |
| | | | proliferation |
| BECN1 | Bcl-2-interacting protein beclin-1 | | Cell cycle regulation and |
| | | | proliferation |
| BRCA1 | Breast cancer 1, early onset; Breast cancer type I | | Cell cycle regulation and |
| | susceptibility protein | | proliferation |
| BRCA2 | Breast cancer 2, early onset; Breast cancer type II | | Cell cycle regulation and |
| | susceptibility protein | | proliferation |
| BRF1 | BRF1 homolog | Hereditary phenotype | |
| BSG | Basigin; Extracellular matrix metalloproteinase | | Protein processing and |
| | inducer (EMMPRIN); Tumor collagenase stimulatory | | turnover, incl. synthesis, |
| | factor (TCSF); CD147 | | cleavage, stabilization and |
| | | | transport, proteolysis |
| CAD | Carbamoyl-phosphate synthetase 2, aspartate trans- | Hereditary phenotype | |
| | carbamylase, and dihydroorotase | | |
| CAV1 | Caveolin-1; Caveolae protein, 22 k | Mesenchymal phenotype | |
| CBX3 | Chromobox homolog 3 | Hereditary phenotype | Cell structure |
| CCND1 | Cyclin D1; Parathyroid adenomatosis 1 (PRAD1); | | Cell cycle regulation and |
| | Bcl-1 | | proliferation |
| CCNE1 | Cyclin E1 | | Cell cycle regulation and |
| | | | proliferation |
| CD36 | CD36 antigen; Collagen type I receptor; | Basal/myoepithelial | Adhesion |
| | Thrombospondin receptor | phenotype | |
| CD44 | CD44 antigen; Hyaluronate receptor; | Mesenchymal phenotype | Adhesion |
| | Hermes antigen gp90 homing receptor | | |
| CDH1 | Cadherin-1; E-cadherin (epithelial); Uvomorulin | Luminal epithelial | Adhesion |
| | | phenotype | |
| CDH11 | Cadherin-11; OB-cadherin (osteoblast) | Mesenchymal phenotype | Adhesion |
| CDH13 | Cadherin-13; H-cadherin (heart) | Mesenchymal phenotype | Adhesion |
| CDK4 | Cyclin dependent kinase 4 | Hereditary phenotype | Cell cycle regulation and |
| | | | proliferation |
| CDKN1A | Cyclin-dependent kinase inhibitor 1A; p21/waf1/cip1 | | Cell cycle regulation and |
| | | | proliferation |
| CDKN1B | Cyclin-dependent kinase inhibitor 1B; p27/kip1 | | Cell cycle regulation and |
| | | | proliferation |
| CDKN1C | Cyclin-dependent kinase inhibitor 1C; p57/waf2 | | Cell cycle regulation and |
| | | | proliferation |
| CDKN2A | Cyclin-dependent kinase inhibitor 2A; p16/ink4/mts1 | | Cell cycle regulation and |
| | | | proliferation |
| CEACAM5 | Carcinoembryonic antigen-related Adhesion molecule | | Adhesion |
| | 5; CD66e | | |
| COX6C | Cytochrome c oxidase subunit VIc | Hereditary phenotype | |
| CSDA | Cold shock domain protein A | Hereditary phenotype | |
| CSF1 | Colony stimulating factor-1; Macrophage-colony | | Inflammatory response |
| | stimulating factor (M-CSF, M-CSF1) | | |
| CSF1R | Colony stimulating factor-1 receptor; | | Inflammatory response |
| | c-fms-encoded protein | | |
| CST6 | Cystatin E/M | | Protein processing and |
| | | | turnover, incl. synthesis, |
| | | | cleavage, stabilization and |
| | | | transport, proteolysis |
| CSTA | Cystatin A; Stefin A | | Protein processing and |
| | | | turnover, incl. synthesis, |
| | | | cleavage, stabilization and |
| | | | transport, proteolysis |
| CTNNB1 | Catenin (cadherin-associated protein), beta 1 (88 kD) | | Adhesion |
| CTPS | CTP synthase | Hereditary phenotype | |
| CTSB | Cathepsin B | Mesenchymal phenotype | Protein processing and |
| | | | turnover, incl. synthesis, |
| | | | cleavage, stabilization and |
| | | | transport, proteolysis |
| CTSD | Cathepsin D | | Protein processing and |
| | | | turnover, incl. synthesis, |
| | | | cleavage, stabilization and |
| | | | transport, proteolysis |
| CTSL | Cathepsin L | Mesenchymal phenotype | Protein processing and |
| | | | turnover, incl. synthesis, |
| | | | cleavage, stabilization and |
| | | | transport, proteolysis |
| CX3CL1 | Chemokine (C-X3-C motif) ligand 1; Small inducible | Basal/myoepithelial | |
| | cytokine subfamily D (Cys-X3-Cys), member 1; | phenotype | |
| | Fractalkine; Neurotactin | | |
| CYP19 | Cytochrome P450, subfamily XIX; Aromatase; | | Oxidative metabolism |
| | Estrogen synthetase | | |
| D123 | D123 gene product | Hereditary phenotype | Cell cycle regulation and |
| | | | proliferation |
| ECGF1 | Endothelial cell growth factor 1, platelet-derived (PD- | | Angiogenesis |
| | ECGF); Thymidine phosphorylase (TP) | | |
| EGFR | Epidermal growth factor receptor | Mesenchymal phenotype | Cell cycle regulation and |
| | | | proliferation |
| EIF4E | Eukaryotic translation initiation factor 4E (EIF4-E); | | Protein processing and |
| | Cap-binding protein | | turnover, incl. synthesis, |
| | | | cleavage, stabilization and |
| | | | transport, proteolysis |
| EMS1 | Cortactin; Amplaxin | | Adhesion |
| ERBB2 | c-erbB-2; Herstatin (HER-2); Neu | ErbB2 phenotype | |
| ESR1 | Estrogen receptor 1; Estrogen receptor-alpha | Luminal epithelial phenotype; | |
| | | Hormonal | |
| | | phenotype | |
| ESR2 | Estrogen receptor 2; Estrogen receptor-beta | Hormonal phenotype | |
| FABP4 | Fatty acid binding protein 4, adipocyte | Basal/myoepithelial | |
| | | phenotype | |
| FGF2 | Fibroblast growth factor 2; | | Angiogenesis |
| | Fibroblast growth factor, basic (bFGF) | | |
| FGF8 | Fibroblast growth factor-8 (androgen-induced) | Mesenchymal phenotype | Angiogenesis |
| FGFR1 | FGF receptor-1; Fms-related tyrosine kinase 2 | | Angiogenesis |
| FHIT | Fragile histidine triad; Bis(5′-adenosyl) triphosphatase; | | Cell cycle regulation and |
| | Diadenosine triphosphate (Ap3A) hydrolase | | proliferation |
| FIGF | c-fos induced growth factor; | | Angiogenesis |
| | Vascular endothelial growth factor D (VEGFD) | | |
| FLT1 | Fms-related tyrosine kinase 1; VEGF receptor 1 | | Angiogenesis |
| FLT4 | Fms-related tyrosine kinase 4; VEGF receptor 3 | | Angiogenesis |
| FOXA1 | Forkhead box A1; Hepatocyte nuclear factor 3-alpha | Luminal epithelial | |
| | (HNF3A) | phenotype | |
| FOXM1 | Forkhead box M1 | Hereditary phenotype | Cell cycle regulation and |
| | | | proliferation |
| G22P1 | Thyroid autoantigen 70 kDa | Hereditary phenotype | |
| GATA3 | GATA binding protein 3 | Luminal epithelial | |
| | | phenotype | |
| GD12 | GDP dissociation inhibitor 2 | Hereditary phenotype | |
| GJA1 | Gap junction protein, alpha 1, 43 kD; Connexin 43 | | Adhesion |
| | (Cx43) | | |
| GJB2 | Gap junction protein, beta 2, 26 kD; Connexin 26 | | Adhesion |
| | (Cx26) | | |
| GNAI3 | Guanine nucleotide binding protein (G protein), alpha | Hereditary phenotype | |
| | inhibiting activity polypeptide 3 | | |
| GPX4 | Glutathione peroxidase 4 | Hereditary phenotype | |
| GRB7 | Growth factor receptor-bound protein 7 | ErbB2 phenotype | |
| GSN | Gelsolin | | Cell structure |
| GSTP1 | Glutathione-S-transferase P(i)1 | Mesenchymal phenotype | Chemoresistance |
| HADHA | Hydroxyacyl-Coenzyme A | Hereditary phenotype | |
| HGF | Hepatocyte growth factor; Scatter factor (SF); | | Angiogenesis |
| | Hepapoietin A | | |
| HSPC195 | Hypothetical protein HSPC195 | Hereditary phenotype | |
| IBSP | Integrin-binding sialoprotein; Bone sialoprotein (BSP) | | Adhesion |
| ICAM1 | Intercellular adhesion molecule-1; Rhinovirus receptor; | Mesenchymal phenotype | Adhesion |
| | CD54 antigen | | |
| IGF2 | Insulin-like growth factor 2; Somatomedin A | Luminal epithelial | |
| | | phenotype | |
| IL11 | Interleukin 11; Adipogenesis inhibitory factor (ADIF) | Mesenchymal phenotype | Inflammatory response |
| IL1A | Interleukin 1, alpha | | Inflammatory response |
| IL1B | Interleukin 1, beta | Mesenchymal phenotype | Inflammatory response |
| IL6 | Interleukin 6; Interferon, beta 2 | Mesenchymal phenotype | Inflammatory response |
| IL8 | Interleukin-8; Monocyte-derived neutrophil-activating | Mesenchymal phenotype | Angiogenesis |
| | protein (MONAP); Monocyte-derived neutrophil | | |
| | chemotactic factor (MDNCF) | | |
| ILF2 | Interleukin enhancer binding factor 2 | Hereditary phenotype | |
| ING1 | Inhibitor of growth 1 family, member 1; | | Cell cycle regulation and |
| | p33ING1 protein | | proliferation |
| ITGA6 | Integrin, alpha 6 | | Adhesion |
| ITGAV | Integrin, alpha V; Vitronectin receptor alpha | | Adhesion |
| | polypeptide; CD51 antigen | | |
| ITGB1 | Integrin, beta 1; Fibronectin receptor, beta polypeptide; | Mesenchymal phenotype | Adhesion |
| | CD29 antigen | | |
| ITGB3 | Integrin, β-3; Platelet glycoprotein IIIa; CD61 antigen | | Adhesion |
| ITGB8 | Integrin, β-8 | Hereditary phenotype | Adhesion |
| KAI1 | Kangai-1; Suppression of tumorigenicity 6 (ST6); | | Cell cycle regulation and |
| | CD82 antigen | | proliferation |
| KDR | Kinase insert domain receptor; VEGF receptor 2; | | Angiogenesis |
| | Flk-1 protein | | |
| KIAA0601 | KIAA0601 protein | Hereditary phenotype | |
| KISS1 | Kiss-1 metastasis suppressor | | Cell cycle regulation and |
| | | | proliferation |
| KLK3 | Kallikrein-3; Prostate-specific antigen (PSA) | | Protein processing and |
| | | | turnover, incl. synthesis, |
| | | | cleavage, stabilization and |
| | | | transport, proteolysis |
| KRT17 | Keratin 17 | Basal/myoepithelial | Cell structure |
| | | phenotype | |
| KRT18 | Keratin 18 | Luminal epithelial | Cell structure |
| | | phenotype | |
| KRT19 | Keratin 19 | Luminal epithelial | Cell structure |
| | | phenotype | |
| KRT5 | Keratin 5; Epidermolysis bullosa simplex, Dowling- | Basal/myoepithelial | Cell structure |
| | Meara/Kobner/Weber-Cockayne types | phenotype | |
| KRT8 | Keratin 8 | Luminal epithelial phenotype; | Cell structure |
| | | Hereditary | |
| | | phenotype | |
| LAMC2 | Laminin, gamma 2 | Basal/myoepithelial | Cell adhesion |
| | | phenotype | |
| LAP18 | Leukemia-associated phosphoprotein p18; Oncoprotein | | Cell cycle regulation and |
| | 18 (OP 18); Stathmin | | proliferation |
| LIV-1 | LIV-1 protein, estrogen regulated | Hormonal phenotype | |
| LRP1 | Low density lipoprotein-related protein 1 | Hereditary phenotype | Cell cycle regulation and |
| | | | proliferation |
| MCAM | Melanoma Adhesion molecule (MCAM); MUC18 | | Adhesion |
| | glycoprotein; CD166 antigen | | |
| MCM7 | Minichromosome maintenance deficient 7 | Hereditary phenotype | |
| MDM2 | Mouse double minute 2, human homolog of; | | Cell cycle regulation and |
| | p53 binding protein | | proliferation |
| MET | Met-protooncogene product; Hepatocyte growth | Mesenchymal phenotype | Angiogenesis |
| | factor receptor | | |
| MGB1 | Mammaglobin-1 | | Cell cycle regulation and |
| | | | proliferation |
| MKI67 | Ki-67 antigen; Mib-1 antigen | | Cell cycle regulation and |
| | | | proliferation |
| MMP1 | Matrix metalloproteinase-1; | Mesenchymal phenotype | Protein processing and |
| | Interstitial collagenase | | turnover, incl. synthesis, |
| | | | cleavage, stabilization and |
| | | | transport, proteolysis |
| MMP11 | Matrix metalloproteinase-11; | Mesenchymal phenotype | Protein processing and |
| | Stromelysin 3 | | turnover, incl. synthesis, |
| | | | cleavage, stabilization and |
| | | | transport, proteolysis |
| MMP13 | Matrix metalloproteinase-13; | | Protein processing and |
| | Collagenase 3 | | turnover, incl. synthesis, |
| | | | cleavage, stabilization and |
| | | | transport, proteolysis |
| MMP14 | Matrix metalloproteinase-14 (membrane-inserted); | Mesenchymal phenotype | Protein processing and |
| | Membrane-type matrix metalloproteinase 1 (MT1- | | turnover, incl. synthesis, |
| | MMP) | | cleavage, stabilization and |
| | | | transport, proteolysis |
| MMP15 | Matrix metalloproteinase-15 (membrane-inserted); | | Protein processing and |
| | Membrane-type matrix metalloproteinase 2 (MT2- | | turnover, incl. synthesis, |
| | MMP) | | cleavage, stabilization and |
| | | | transport, proteolysis |
| MMP2 | Matrix metalloproteinase-2; Gelatinase A; | Mesenchymal phenotype | Protein processing and |
| | 72 kD-gelatinase | | turnover, incl. synthesis, |
| | | | cleavage, stabilization and |
| | | | transport, proteolysis |
| MMP3 | Matrix metalloproteinase-3; Stromelysin 1 | | Protein processing and |
| | | | turnover, incl. synthesis, |
| | | | cleavage, stabilization and |
| | | | transport, proteolysis |
| MMP7 | Matrix metalloproteinase-7; Matrilysin | Mesenchymal phenotype | Protein processing and |
| | | | turnover, incl. synthesis, |
| | | | cleavage, stabilization and |
| | | | transport, proteolysis |
| MMP9 | Matrix metalloproteinase-9; Gelatinase B; 92 kD- | | Protein processing and |
| | gelatinase | | turnover, incl. synthesis, |
| | | | cleavage, stabilization and |
| | | | transport, proteolysis |
| MTMR4 | Myotubularin related protein 4 | Hereditary phenotype | |
| MUC1 | Mucin-1, transmembrane; CA15-3 antigen; | Luminal epithelial | Adhesion |
| | Episialin; Polymorphic epithelial mucin (PEM); | phenotype | |
| | Epithelial membrane antigen (EMA); | | |
| MVP | Major vault protein; Lung resistance protein (LRP) | | Chemoresistance |
| MX2 | Myxovirus (influenza virus) resistance 2 | Hereditary phenotype | |
| MYC | V-myc avian myelocytomatosis viral oncogene | | Cell cycle regulation and |
| | homolog | | proliferation |
| NCOA1 | Nuclear receptor coactivator 1; | Hormonal phenotype | |
| | Steroid receptor coactivator 1 (SRC-1) | | |
| NCOA2 | Nuclear receptor coactivator 2; Steroid receptor | Hormonal phenotype | |
| | coactivator 2 (SRC-2); Transcriptional intermediary | | |
| | factor 2 (TIF2); Glucocorticoid receptor interacting | | |
| | protein 1 (GRIP1) | | |
| NCOA3 | Nuclear receptor coactivator 3; Steroid receptor coactivator | Hormonal phenotype | |
| | 3; Amplified in Breast Cancer (AIB1); Thyroid | | |
| | hormone receptor activator molecule (TRAM-1); | | |
| | Receptor-associated coactivator 3 (RAC3) | | |
| NCOR1 | Nuclear receptor co-repressor 1; KIAA1047 protein | Hormonal phenotype | |
| NCOR2 | Nuclear receptor co-repressor 2; Silencing mediator of | Hormonal phenotype | |
| | retinoid and thyroid hormone action (SMRT) | | |
| NIFU | Nitrogen fixation cluster-like | Hereditary phenotype | |
| NME1 | Non-metastatic cells 1, protein expressed in; Nm23-h1, | | Cell cycle regulation and |
| | nm23A; Nucleoside diphosphate kinase A (NDKA); | | proliferation |
| NME2 | Non-metastatic cells 2, protein expressed in; Nm23-h2, | | Cell cycle regulation and |
| | nm23B; Nucleoside diphosphate kinase B (NDKB); | | proliferation |
| NSPEP1 | Nuclease sensitive element binding protein 1 | Hereditary phenotype | |
| ODC1 | Ornithine decarboxylase 1 | Hereditary phenotype | Cell cycle regulation and |
| | | | proliferation |
| PAI-RBP1 | PAI-1 mRNA-binding protein | Hereditary phenotype | |
| PCNA | Proliferating cell nuclear antigen | Hereditary phenotype | Cell cycle regulation and |
| | | | proliferation |
| PDGFB | Platelet-derived growth factor beta polypeptide | Hereditary phenotype | |
| PDZK1 | PDZ domain containing 1 | Hormonal phenotype | |
| | | Luminal epithelial | |
| | | phenotype | |
| PFKP | Phosphofructokinase, platelet | Hereditary phenotype | |
| PGR | Progesterone receptor | Hormonal phenotype | |
| | | Luminal epithelial | |
| | | phenotype | |
| PHYH | Phytanoyl-CoA hydroxylase | Hereditary phenotype | |
| PIP | Prolactin-induced protein; | Luminal epithelial | |
| | Gross cystic disease fluid protein 15 (GCDFP-15) | phenotype | |
| PLAT | Plasminogen activator, tissue-type (tPA) | Mesenchymal phenotype | Protein processing and |
| | | | turnover, incl. synthesis, |
| | | | cleavage, stabilization and |
| | | | transport, proteolysis |
| PLAU | Plasminogen activator, urokinase (uPA) | Mesenchymal phenotype | Protein processing and |
| | | | turnover, incl. synthesis, |
| | | | cleavage, stabilization and |
| | | | transport, proteolysis |
| PLAUR | Plasminogen activator, urokinase receptor (uPAR); | | Protein processing and |
| | CD87 antigen | | turnover, incl. synthesis, |
| | | | cleavage, stabilization and |
| | | | transport, proteolysis |
| PPP1CB | Protein phosphatase 1, catalytic subunit, beta isoform | Hereditary phenotype | |
| PTGS2 | Prostaglandin-endoperoxide synthase 2; Prostaglandin | Mesenchymal phenotype | Inflammatory response |
| | G/H synthase; Cyclooxygenase-2 (COX-2) | | |
| PTHLH | Parathyroid hormone-like hormone; | | Cell cycle regulation and |
| | Parathyroid hormone-related protein | | proliferation |
| PTN | Pleiotrophin; Heparin binding growth factor 8; | Basal/myoepithelial | Cell cycle regulation and |
| | Neurite growth-promoting factor 1 | phenotype | proliferation |
| RB1 | Retinoblastoma-1 | | Cell cycle regulation and |
| | | | proliferation |
| RBL2 | Retinoblastoma-like 2 | Hereditary phenotype | |
| S100A4 | S100 calcium-binding protein A4; Metastasin; | | Cell cycle regulation and |
| | Placental calcium-binding protein (CAPL) | | proliferation |
| SELENBP1 | Selenium binding protein 1 (SBP1) | Luminal epithelial | |
| | | phenotype | |
| SERPINB2 | Serine (or cysteine) proteinase inhibitor, clade B, | | Protein processing and |
| | member 2; Plasminogen activator inhibitor, type II | | turnover, incl. synthesis, |
| | (PAI-2) | | cleavage, stabilization and |
| | | | transport, proteolysis |
| SERPINB5 | Serine (or cysteine) proteinase inhibitor, clade B, | Basal/myoepithelial | Protein processing and |
| | member 5; Protease inhibitor 5 (P15); Maspin | phenotype | turnover, incl. synthesis, |
| | | | cleavage, stabilization and |
| | | | transport, proteolysis |
| SERPINE1 | Serine (or cysteine) proteinase inhibitor, clade E, | Mesenchymal phenotype | Protein processing and |
| | member 1; Plasminogen activator inhibitor, type I | | turnover, incl. synthesis, |
| | (PAI-1) | | cleavage, stabilization and |
| | | | transport, proteolysis |
| SLPI | Secretory leukocyte protease inhibitor; | Basal/myoepithelial | Protein processing and |
| | Antileukoproteinase | phenotype | turnover, incl. synthesis, |
| | | | cleavage, stabilization and |
| | | | transport, proteolysis |
| SOD2 | Superoxide dismutase-2, mitochondrial; Manganese- | | Angiogenesis |
| | containing superoxide dismutase (MnSOD) | | |
| SPHAR | S-phase response | Hereditary phenotype | Cell cycle regulation and |
| | | | proliferation |
| SPRR1A | Small proline-rich protein 1A | | Cell structure |
| SPS | Selenophosphate synthetase | Hereditary phenotype | |
| SRA1 | Steroid receptor RNA activator (1) | Hormonal phenotype | |
| ST13 | Suppression of tumorigenicity 13 | Hereditary phenotype | |
| STAB1 | Stabilin 1 | Hereditary phenotype | |
| STARD3 | START domain containing 3; MLN 64 protein | ErbB2 phenotype | |
| | (MLN64); Steroidogenic acute regulatory protein | | |
| | related | | |
| STC2 | Stanniocalcin 2 | Hormonal phenotype | |
| TFAP2C | Transcription factor AP-2 gamma | Hereditary phenotype | |
| TFF1 | Trefoil factor 1; pS2; Breast cancer, estrogen-inducible | Hormonal phenotype | |
| | sequence expressed in (BCEI) | Luminal epithelial | |
| | | phenotype | |
| TFF3 | Trefoil factor 3; Intestinal trefoil factor | Hormonal phenotype | |
| | | Luminal epithelial | |
| | | phenotype | |
| THBS1 | Thrombospondin-1 | Mesenchymal phenotype | Angiogenesis |
| THBS2 | Thrombospondin-2 | | Angiogenesis |
| TIMP1 | Tissue inhibitor of metalloproteinase-1; | Mesenchymal phenotype | Protein processing and |
| | Erythroid potentiating activity (EPA) | | turnover, incl. synthesis, |
| | | | cleavage, stabilization and |
| | | | transport, proteolysis |
| TIMP2 | Tissue inhibitor of metalloproteinase-2 | | Protein processing and |
| | | | turnover, incl. synthesis, |
| | | | cleavage, stabilization and |
| | | | transport, proteolysis |
| TJP1 | Tight junction protein-1; | Luminal epithelial | Adhesion |
| | Zonula occludens 1 protein (ZO-1) | phenotype | |
| TMSB10 | Thymosin, beta 10 | | Cell cycle regulation and |
| | | | proliferation |
| TNF | Tumor necrosis factor alpha | | Angiogenesis |
| TNFRSF11A | Tumor necrosis factor receptor superfamily, member | | Cell cycle regulation and |
| | 11A | | proliferation |
| TNFRSF11B | Tumor necrosis factor receptor superfamily, member | | Cell cycle regulation and |
| | 11B | | proliferation |
| TNFSF11 | Tumor necrosis factor superfamily, member 11 | | Cell cycle regulation and |
| | | | proliferation |
| TOB1 | Transducer of ERBB2, 1 | Hereditary phenotype | Cell cycle regulation and |
| | | | proliferation |
| TOP2A | Topoisomerase (DNA) II alpha (170 kD) | | Chemoresistance |
| TP53 | Tumor protein p53 | | Cell cycle regulation and |
| | | | proliferation |
| TP53BP2 | Tumor protein p53 binding protein, 2 | Hereditary phenotype | |
| UGTREL1 | UDP-galactose transporter related | Hereditary phenotype | |
| VEGF | Vascular endothelial growth factor; | | Angiogenesis |
| | Vascular permeability factor (VPF) | | |
| VEGFB | Vascular endothelial growth factor B | | Angiogenesis |
| VEGFC | Vascular endothelial growth factor C | Mesenchymal phenotype | Angiogenesis |
| VIM | Vimentin | Mesenchymal phenotype | Cell structure |
| VLDLR | Very low density lipoprotein receptor | Hereditary phenotype | |
| VWF | Von Willebrand factor | | Angiogenesis |
| XBP1 | X-box binding protein 1 | Hormonal phenotype | |
| | | Luminal epithelial | |
| | | phenotype | |
| ZNF22 | Zinc finger protein 22 | Hereditary phenotype | |
| ZNF161 | Zinc finger protein 161 | Hereditary phenotype | |
| RPL13A | 23 KDa Highly basic protein | | House keeping gene |
| ALDOA | Aldolase A, fructose biphosphate | | House keeping gene |
| K-ALPHA-1 | Alpha-tubulin | | House keeping gene |
| ACTB | Beta-Actin | | House keeping gene |
| PPIE | Cyclophilin 33A | | House keeping gene |
| GAPD | Glyceraldehyde-3-phosphate-dehydrogenase | | House keeping gene |
| HK1 | Hexokinase 1 | | House keeping gene |
| HPRT1 | Hypoxanthine phosphoribosyltransferase 1 | | House Keeping gene |
| MDH1 | Malate dehydrogenase 1 | | House keeping gene |
| YWHAZ | Phospholipase A2 | | House keeping gene |
| RPS9 | Ribosomal Proteine S9 | | House keeping gene |
| SDS | Serine Dehydratase | | House keeping gene |
| TFRC | Transferrin receptor | | House keeping gene |

Due to the sequencing of the human genome and the publication of the gene map the above mentioned examples are known to the skilled person and appropriate capture probes may be designed on the basis of such information. Likewise, additional genes comprised by the above categories are known to the skilled person and may be derived e.g. from public databases like the National Center for Biotechnology Information (NCBI), the LocusLink web site (http://www.ncbi.nlm.nih.gov/LocusLink/) or the GeneCards Encyclopedia (http://bioinformatics.weizmann.ac.il/cards/). LocusLink provides a single query interface to curated sequence and descriptive information about genetic loci. It presents information on official nomenclature, aliases, sequence accessions, phenotypes, EC numbers, MIM numbers, UniGene clusters, homology, map locations, and related web sites. The GeneCards Encyclopedia integrates a subset of the information stored in major data sources dealing with human genes and their products (with a major focus on medical aspects).

Additional features and advantages of the present invention are described in, and will be apparent from, the following Detailed Description of the Invention and the figures.

### BRIEF DESCRIPTION OF THE FIGURES

FIG. 1 is a schematic presentation of a pattern of a microarray for the quantification of differentiated breast cancer gene expression with appropriated controls.

### DETAILED DESCRIPTION OF THE INVENTION

The present inventors have found that in order to provide a proper and reliable diagnosis and prognosis of breast cancer it is not enough to just simply classify a tumor according to genes known to be involved, but that also additional information about the present cellular status of the cell is required. Only this entire information allows the attending physician to apply an appropriate regime for the particular type of cancer, to give a reliable prognosis and eventually to monitor the success of the treatment. Basically, the present inventors provide a characterization of the biological and the pathological aspect of a breast tumor based on the quantification of a minimal number of genes covering 6 phenotypes and other several cellular functions which allow to class the tumors and cell lines according to the expected cell tumor origin and their biological characteristic.

In the present invention the term “cellular function” means a function which is essential in order to obtain an overview of the modifications occurring in the “vital” cellular functions under specific biological conditions. “Vital” functions are functions which are essential for life, division and growth of the cells. Examplarily mentioned “cellular functions” are mentioned in table I, supra.

The term “expressed genes” are the parts of the genomic DNA which are transcribed into mRNA and then translated into a peptides or proteins. The measurement of the expressed genes is performed on either molecules within this process most currently the detection of the mRNA or of the peptide or protein. The detection can also be based on specific property of the protein being for example its enzymatic activity.

The terms “nucleic acid, array, probe, target nucleic acid, bind substantially, hybridizing specifically to, background, quantifying” are as described in the international patent application WO97/273 17, which is incorporated herein by reference.

The term “nucleotide triphosphate” refers to nucleotides present in either as DNA or RNA and thus includes nucleotides which incorporate adenine, cytosine, guanine, thymine and uracil as bases, the sugar moieties being deoxyribose or ribose. Other modified bases capable of base pairing with one of the conventional bases adenine, cytosine, guanine, thymine and uracil may be employed. Such modified bases include for example 8-azaguanine and hypoxanthine.

The term “nucleotide” as used herein refers to nucleosides present in nucleic acids (either DNA or RNA) compared with the bases of said nucleic acid, and includes nucleotides comprising usual or modified bases as above described.

References to nucleotide(s), polynucleotide(s) and the like include analogous species wherein the sugar-phosphate backbone is modified and/or replaced, provided that its hybridization properties are not destroyed. By way of example the backbone may be replaced by an equivalent synthetic peptide, called Peptide Nucleic Acid (PNA).

The terms “nucleotide species” is a composition of related nucleotides for the detection of a given sequence by base pairing hybridization; nucleotides are synthesized either chemically or enzymatically but the synthesis is not always perfect and the main sequence is contaminated by other related sequences like shorter one or sequences differing by a one or a few nucleotides. The essential characteristic of one nucleotides species for the invention being that the overall species can be used for capture of a given sequence belonging to a given gene.

“Polynucleotide” sequences that are complementary to one or more of the genes described herein, refers to polynucleotides that are capable of hybridizing under stringent conditions to at least part of the nucleotide sequence of said genes. Polynucleotides also include oligonucleotides which can be used under particular conditions; such hybridizable polynucleotides will typically exhibit at least about 75% sequence identity at the nucleotide level to said genes, preferably about 80% or 85% sequence identity or more preferably about 90% or 95% or more nucleotide sequence identity to said genes. They are composed of either small sequences typically 15-30 base long or longer ones being between 30 and 100 or even longer, between 100 and 300 bases long.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The term “capture probe” designates a molecule which is able to specifically binds to a given polynucleotide or polypeptide. Polynucleotide binding is obtained through base pairing between two polynucleotides, one being the immobilized capture probe and the other one the target to be detected. Polypeptide binding is best performed using antibodies specific of the polypeptide for the capture of a given polypeptide or protein. Part of the antibodies, or recombinant proteins incorporating part of the antibodies, typically the variable domains, or even proteins being able to specifically recognized the peptide can also be used as capture probes.

The terms “background” or “background signal intensity” refers to hybridization signals resulting from non-specific binding, or other interactions, between the labeled target nucleic acids and components of the polynucleotide array (e. g., the polynucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated individually for each spot, being the level intensity of the signal around each of the spot.

The phrase “hybridizing specifically to” refers to the binding, duplexing or hybridizing of a molecule substantially to or only to a particular nucleotide sequence or sequences under stringent conditions when that sequence is present in a complex mixture (e. g., total cellular) DNA or RNA.

The “hybridized nucleic acids” are typically detected by detecting one or more “labels” attached to the sample nucleic acids. The labels may be incorporated by any of a number of means well known to those of skill in the art, such as detailed in WO 99/32660, which is incorporated herein by way of reference.

The term “capture probes” in the sense of the present invention shall designate genes or parts of genes of different length, e.g. between 10 and 1500 nucleotides, which are either synthesized chemically in situ on the surface of the support or laid down thereon. Moreover, this term shall also designate polypeptides or fragments thereof, or antibodies directed to particular polypeptides, which terms are used interchangeably, attached or adsorbed on the support.

During the extensive studies leading to the present invention it has been found that it is in effect possible to provide a tool for diagnostic, prognostic and the curative follow up of breast tumors by the quantification of the expression of specifically selected genes, or their products, according to 6 particular phenotypes, namely the luminal/epithelial phenotype, the basal/myoepithelial phenotype, the mesenchymal phenotype, the ErbB2 phenotype, the hormonal phenotype and the hereditary susceptibility representing a molecular signature characteristic of the breast tumor, and taken together with some other less specific functions related to cellular functions, e.g. to cell adhesion, cell cycle regulation, chemoresistance, angiogenesis, protein processing and turnover, oxidative metabolism, inflammatory response and cell structure and/or some house-keeping genes.

None of the prior art documents mentions the necessity to investigate the different cellular functions related to cancer cell phenotypes together with specific cellular gene functions and house keeping gene quantification to arrive at a reliable and simple diagnostic tool.

In general, the differentiated gene expression of genes associated with at least 4 or 5 of the 6 phenotypes gives a good representation of the tumor status and of the characteristics of the cells for a diagnostic, prognostic and therapeutic implication for the medical treatment of the tumors and of the patient. Specifically, the luminal/epithelial phenotypes characterize cells from the upper layer of mammary epithelium; the basal/myoepithelial phenotype are characteristic for cells present on the lower layer of the epithelium and give rise to other cell type including myoepithelial cells. The mesenchymal phenotype is characterized of cells of the mesenchyme and the mesenchymal phenotype is represented into carcinosarcoma. On the other side, ErbB2 phenotype resulting from ErbB2 amplification is characterized by aggressiveness and poor prognostic of the patient outcome. It can be present or not on the other cell phenotypes. The hormone phenotype is the presence or not of the steroid hormone receptors and the other proteins involved into the transfer of the information into biochemical and gene expression activation and repression due to the presence of hormone. The estrogen receptor-alpha (ER-alpha, gene ESR1), is one of the best representative of such receptor phenotype family with the other members described in table I. The hormone phenotype considered as ER+ is a prognostic indicator which expression is associated with a longer survival of patients and a predictor of patient responsiveness to anti-estrogens, particularly in node-negative patients (cf. Valavaara R. Oncology (Huntingt) 11 (1997), 14-8). The hereditary susceptibility is mostly due to mutations in some specific genes which increases the predispositon risk and necessitates adapted medical care. It constitutes in itself a particular phenotype since it gives information on the possible origin of the cancer or to the susceptibility of the patient to the cancer occurring due to mutations in the genomic DNA of the cells.

In one embodiment the hormone phenotype is also associated with the expression of mRNA encoded by TFF1, CCND1, PGR, MYC, which are relevant for the transcriptional functionality of the ER.

Exemplarily named other genes that may serve as clinical indicators in breast cancer cells are keratin 19 (gene KRT19), parathyroid hormone-related peptide (PTHLH), interleukin-6 (IL6), vascular endothelial growth factor (VEGF) and bcl-2 (BCL2).

E.g., the gene expression changes associated with the hereditary phenotype is linked to mutations occurring in specific genes including the BRCA1 and/or BRCA2 (Hedenfalk et al. N Engl J Med 344 (2001), 539-48). The gene expression pattern is linked to allele mutant conferring the patient a predisposition to ovarian cancer and may be linked to an increase risk for ovarian and pancreatic cancer. Risk of cancer for the phenotype positive patients is also influenced by other environment effect such as hormones which will modulate the hereditary susceptibility for cancer. In another embodiment the gene expression obtained in the hereditary phenotype allows the classification of the tumors according to the hereditary and the sporadic forms of cancers. In a more precise analysis, gene expression changes are associated specifically with the BRCA 1 mutations and some other with the BRCA 2 mutations. The gene expression changes associated with the BRAC1 mutations are part of the group of KRT8, VLDLR, MCM7, BRF1, SPS. And the gene expression changes associated with the BRCA 2 mutations are part of the group containing ACTRIA, PCNA, UGTREL1, ZNF161, ARVCF, PDGFB, PPPICB. Other genes like MCH2, CTGF and PDCD5 are also useful to discriminate between the BRCA1 and BRCA2 mutations.

As mentioned above, the inventors have found that apart from knowledge about any of the above phenotypes a minimum information about other vital cellular functions are required to obtain a general picture of the cell and to give a reliable and useful diagnostic and/or prognostic of cell tumor. The additional information obtained from the cellular functions allows a skilled person to better understand how the tumor cell has been altered. Cellular functions relate to the vitality, defense, metabolism, apoptose, cell division, cell response to cytokine or growth factors and other basic cellular properties of a given cell. On the other hand, the phenotype is characteristic of the tumor cell. The information obtained from the alteration of the cellular functions would indicate how the function has been altered in relation with the tumor phenotype. Hence, modifications of cellular functions are important to better characterize tumors (diagnostic markers), to foresee the evolution and the complications of tumors (prognostic markers) and provide an estimation of the patient responsiveness to specific therapy (predictive markers).

Some phenotypes or protein functions are important for monitoring patient treatment. E.g. in case the estrogen receptor is present treatment with anti-estrogen drugs will be applied. The level of proliferative activity is also useful to determine the sensitivity to anti-proliferative drugs. The chemoresistance activated will also influence the type of chemotherapeutic agents chosen to be active in the cancer cells. The level of angiogenesis is also a target for anti-angiogenesis drugs. The inhibition of protease of collagenase is also linked to the proteolysis activity of the cells. The adhesion is important to estimate the possible level of metastasis. High oxidative metabolism is a usual characteristic of aggressive tumors. Inflammatory cancers are usually aggressive tumors with fort evolution. Some of the genes related to protein processing and turnover are also linked with specific tumors characterization.

From a considerable number of studies eventually resulting in the present invention, it has been found that a clear link exists between the curative outcome of a breast cancer patient and the phenotype of the tumor. This latter relates mainly to the expression of three extended gene signatures (“luminal epithelial”, “basal/myo-epithelial”, “mesenchymal”), to which both, the transformed cells and their normal surrounding cells may contribute and the hereditary phenotype. The phenotype of a tumor is also crucially associated to its expression level of the (hormone) estrogen receptor-alpha (gene ESR1) and the tyrosine kinase ErbB-2 (gene ERBB2). These, and other accompanying molecules, define the “hormonal phenotype” and the “ERBB2-phenotype”, respectively.

On the other hand, specific cellular functions involved in tumor progression, such as proliferation, angiogenesis, protein degradation, have drawn a considerable attention in breast cancer research. From related studies, various function-targeted therapeutic strategies have been designed.

According to an embodiment of the invention, the variations of the gene expression fulfil the following criteria in order to be relevant for interpretation: 1) these variations are recognized as being, or at least suspected to be, of diagnostic, prognostic, or predictive value; 2) they are expressed in tumors at levels detectable by the DNA chip. The present invention gives also a means of providing micro-array with a direct detection of more than 75% of the genes with cDNA prepared from as low as 1 to 10 μg of total RNA; 3) variations of mRNA amount parallel the variations of the corresponding protein and gene expression. The amount of mRNA at a given moment in the cells reflects the equilibrium between transcription and degradation. This amount is also influenced by gene deletions and amplifications (as frequently observed i.e. for genes like ERBB2, MYC, CCND1, EMS1, FGFR1, MDM2). Protein structures and activities are the ultimate support of cell functions and tumor properties. A good correlation between mRNA and protein levels may be crucial when it concerns therapeutic target proteins, such as proteinases, vascular endothelial growth factor (VEGF), c-erbB-2 (ERBB2), or resistance markers such as breast cancer resistance protein (ABCG2), multidrug resistance protein-1 (ABCB1) or the lung resistance protein (MVP). Regarding ER-alpha (ESR1), a simple linear relationship exists between its 6.7-kb mRNA measured by northern blot and the receptor level evaluated by ligand-binding assay (LBA) (Lacroix M et al. Res. Treat. Jun 67(3) (2001), 263-71). The polynucleotide detection is favourably replaces by the polypeptide detection in order to directly obtained the quantification of the transcript of the genes.

In principle the micro-array may contain as few as 16 capture probes, i.e. one capture probe associated with each of the 6 cancer phenotypes and 10 capture probes associated with the cellular functions selected. Yet, the number of capture probes on the micro-array may be selected according to the need of the skilled person and may contain capture probes for the detection of up to about 3000 different genes, e.g. about 100, or 200 or 500 or 1000, or 2000 different genes. Since the capture probes are arranged on the solid support in the form of an array each gene is quantified by spots with a single nucleotide species, wherein one spot is sufficient for the identification and quantification of one gene.

According to a preferred embodiment of the invention capture probes are long polynucleotides and are uniques for each of the genes to be detected and quantified on the array. Long capture probes mean capture probes of 15 to 1000 nucleotides in length, e.g. of 15 to 200, or 15 to 150 nucleotides or 15 to 100, and are fixed on a support being any solid support as long as they are able to hybridized with their corresponding cDNA and be identified and quantified. The density of the capture nucleotide sequences bound to the surface of the solid support may be superior to 3 fmoles per cm of solid support surface.

Direct capture of the cDNA is the preferred embodiment making the data mining and the interpretation of the results easier. The method does not exclude the use of fragmented cDNA or RNA as a means of detection of gene by determining for each gene the pattern of hybridization on oligonucleotides present on the array.

In another embodiment, the capture probes bind the cDNA of the genes close to the 3 poly(A+) region of the corresponding mRNA. Retro-transcription of mRNAs begin at their 3′-poly(A+) region and is not always complete, which indeed gives rise to a population of more or less complete cDNAs. To improve the efficiency of hybridization, it may thus appear preferable to design capture sequences specific to mRNAs regions close to their 3′-end.

The capture probes sequences are preferably chosen so as to avoid cross-reaction with other gene(s). Therefore, regions of high (>50%) homologies between genes, able to yield cross-hybridizations between capture probes and target cDNAs will not be the preferred species.

Also variants of the genes may specifically be detected and quantified on the array by specific capture probes. In some tissues, multiple mRNAs are transcribed from the same gene. These variants often exhibit more or less overlapping sequences. Selection of the capture probes has to be specific for such marker, when all variants do not have the same potential importance as diagnostic, prognostic, or predictive indicator, or when it is recommended to detect only some specific ones. For instance, two integrin alpha 6 (ITGA6) mRNA variants have been identified in breast tumors. They encode proteins differing by their C-terminal cytoplasmic domain. Increased integrin alpha 6 expression could be associated with the metastatic phenotype of breast cancer cells, but this has not been specifically ascribed to any of the variants. Similarly, the potential prognostic value of bcl-2 (BCL2) in breast cancer has not been clearly associated so far to any of its two transcripts, differing by their C-terminus. For ITGA6 as well as for BCL2, it appears thus pertinent to design a capture probe recognizing both variant mRNAs.

At least four variants (A to D) have been found for the integrin beta 1 (ITGB 1), which mediates interactions between cells and the extracellular matrix. The relative amount of these forms is likely to vary in breast tumors. However, preferably the C variant will be detected, which has been shown to inhibit cell proliferation in vitro and is down-regulated in carcinomas. Two capture probes should be designed, one specific to the C form, the other detecting all ITGB 1 variants.

The CD44 glycoprotein is involved in cell-cell and cell-matrix interactions. The CD44 gene contains 20 exons. Exons 1-5 and 16-20 are spliced together to form a transcript that encodes the ubiquitously expressed standard isoform (known as CD44s). The 10 variable exons 6-15 (also named v1-v10) can be alternatively spliced and included within the standard exons at an insertion site between exons 5 and 16, giving rise to a pleiad of so-called CD44v variants. Studies show that not all variants do exhibit the same interest as indicators in breast cancer. Accordingly at least a capture probe is to be designed recognizing the CD44v6 variant, which is a marker to identify node-negative patients with a relatively favorable prognosis. Also the CD44v7-v8 variant, that seems to direct breast tumor cells to lymph nodes and lymphatic vessels, is considered to be a valuable probe.

Another example is MUC1. This gene encodes at least three proteins, Muc1/y, Muc1/sec, Muc1/rep. All Muc1 proteins appear to play a role in reducing cell-cell and integrin-mediated cell-matrix interactions, and probably in the metastatic spread of cancer cells from the initial tumor site. More generally, MUC1 expression seems to enhance tumor initiation and progression. This suggests that the impact of MUC1 on tumor properties depends on the relative levels of its three protein products. It is, therefore of considerably interest to specifically detect the messengers encoding the three proteins Muc1/y, Muc1/sec and Muc1/rep.

After hybridzation with the target nucleotides of the biological sample the spot intensity is read according to the label utilized, e.g. in fluorescence or colorimetry. The quantification of the genes may be performed by standard techniques, e.g. by comparison with internal standards introduced into the retro-transcription of mRNA.

The solid support as such may be made from any material conventionally used for this purpose and is preferably selected from glass, plastic, filters, metals and/or electronic chips.

According to another embodiment the present invention also provides a method for the diagnosis and/or prognosis of breast cancer, which comprises the steps of providing a micro-array as detailed above and quantifying differentially expressed genes, selected from at least 4 of the 6 cancer phenotypes provided on the support, and at least 10 other genes associated with at least 3 cellular functions and/or the house keeping genes.

According to an embodiment the method may be performed on cDNA obtained by retrotranscription from total RNA or mRNA. To this end, total RNA is extracted from tissue and an amount of about 0.1 to 100 μg, preferably 0.1. to 50 μg, more preferably 0.1. to 20 μg, even more preferably 0.1 to 10 μg, or even more preferred between 0.1 and 2 μg is used for direct labeling and hybridization on the array. mRNA may also be processed in the same way with a much lower amount to be used for the copying into cDNA. When RNA is amplified by T7 polymerase based method, PCR, rolling circle or other methods, detection is possible even at lower concentration than 0.1 μg of total RNA or mRNA depending on the amplification obtained being usually in the order of a few hundreds for the T7 polymerase and much higher for the PCR or rolling circle amplifications. In extreme cases, detection of a single cell or a group of a few cells like obtained by laser dissection methods is feasible. In amplification methods, however, different genes are amplified with different efficiencies and corrections have to be provided for the pitfall introduced in the differentially amplified genes.

According to another preferred embodiment the original nucleotide sequences to be detected and/or to be quantified are RNA sequences by the retro-transcription of the 3′ or 5′ end wherein consensus primer and possibly a stopper sequence are used. Preferably, the copied or amplified sequences are detected without previous cutting of original sequences into smaller portions.

The present invention also pertains to a diagnostic and/or prognostic kit, which comprises means and media for performing the above method.

Specifically, a gene expression analysis is performed on the present micro-array by preparing a gene expression profile from cells or from tissues incubated in the presence of drugs or from samples of a patient comprising tumor cells treated e.g. with a drug and comparing the expression profile to a gene expression profile from an untreated cell population comprising breast cancer cells or not. Based on the array identification of gene expression the effect of the drugs or chemical may then be evaluated for its potential activity. Therefore, the present invention also comprises the use of the present micro-array in the treatment of breast cancer.

E.g. a ductal carcinoma may be identified in a patient, comprising detecting the level of expression in a tissue sample of at least one gene associated with each of at least 4, preferably 5 of the 6 cell phenotypes and at least 10 other genes associated with at least 3 functions as listed in table I-II, wherein differential expression of the genes in table II is indicative of ductal carcinoma.

Also, the progression of carcinogenesis in a patient may be determined according to the present invention, comprising detecting the level of expression in a tissue sample of at least one gene associated with each of at least 4, preferably 5 of the 6 cell phenotypes and at least 10 other genes associated with at least 3 functions as listed in table I-II; wherein differential expression of the genes in table II is indicative of breast carcinogenesis.

The hereditary origin or the prognostic of susceptibility to breast tumor cancer is determined by determination of the differentiation of gene expression in breast cells on a microarray bearing the capture probes for at least 4 genes or their products typical of the hereditary phenotype together and at least 4 genes associated to other cellular functions and/or house keeping genes.

In one of the embodiment, the invention provides a method of screening for an agent capable of modulating the onset or progression of breast cancer, comprising the steps of exposing a cell to the agent; and detecting the expression level of at least one gene associated with each of at least 4, preferably 5 of the 6 cell phenotypes and at least 10 other genes associated with at least 3 functions as listed in table I-II.

In another embodiment, the invention further includes computer systems comprising a database containing information identifying the expression level in breast tissue of at least one gene associated with each of the 6 cell phenotypes and at least 10 other genes associated with at least 3 functions as listed in tables I-II and a user interface to view the information. The database may further include sequence information for the genes, information identifying the expression level for the set of genes in normal breast tissue and cancerous tissue and may contain links to external databases such as GenBank. The present invention includes relational databases containing sequence information, for instance for one or more of the genes of Tables I-II, as well as gene expression information in various breast tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. Methods for the configuration and construction of such databases are widely available, for instance, U.S. Pat. No. 5,953,727, which is incorporated herein by reference in its entirety.

According to the present invention, potential drugs can be screened to determine if application of the drug alters the expression of the genes identified herein. This may be useful, for example, in determining whether a particular drug is effective in treating a particular patient with breast cancer. In the case where a gene expression is affected by the potential drug such that its level of expression returns to normal, the drug is indicated in the treatment of breast cancer. Similarly, a drug which causes expression of a gene which is not normally expressed by epithelial cells in the breast, may be contraindicated in the treatment of breast cancer.

Assays to monitor the expression of a marker or markers as e.g. defined in Tables I-II may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

The genes identified as being differentially expressed in breast cancer may be used in a variety of nucleic acid detection assays to detect or quantify the expression level of a gene or multiple genes in a given sample. For example, traditional Northern blotting, nuclease protection, RT-PCR and differential display methods may be used for detecting gene expression levels.

The protein products of the genes identified herein can also be assayed to determine the amount of expression. Methods for assaying for a protein include Western blot, immunoprecipitation, radioimmunoassay and protein chips. Protein chips are supports bearing as capture probes antibodies or related proteins specific of the different proteins or peptides to be analyzed. Antibodies are either on the same support as a protein array or are on different supports as beads, each beads being specific for the detection of proteins. It is preferred, however, that the mRNA be assayed as an indication of expression. Methods for assaying for mRNA include Northern blots, slot blots, dot blots, and hybridization to an ordered array of polynucleotides. Any method for specifically and quantitatively measuring a specific protein or mRNA or DNA product can be used. However, methods and assays of the invention are most efficiently designed with PCR or array or chip hybridization-based methods for detecting the expression of a large number of genes.

Any hybridization assay format may be used, including solution-based and solid support-based assay formats. A preferred solid support is a low density array also known as a DNA chip or a gene chip. In one assay format, the array containing probes to at least one gene associated with each of the 6 cell phenotypes and at least 10 other genes associated with at least 3 functions as e.g. listed in tables I-II, may be used to directly monitor or detect changes in gene expression in the treated or exposed cell as described herein. Assays of the invention may measure the expression levels of about 14, 50, 100, 400, 1000 or 3000 genes with some or all from the table II. The number of genes to be detected is limited in the invention since it allows to better concentrate on gene useful for the characterization of the tumors and to give a more quick and precise response to the questions of the prognostic, diagnostic and therapeutic follow up of the patients. The larger the number of genes to be analyzed and treated for data mining, the less efficient the correlation and the outcome of the analysis.

In another assay format, cells or cell lines are first identified which express one or more of the gene products of the invention physiologically. Cells and/or cell lines so identified would preferably comprise the necessary cellular machinery to ensure that the transcriptional and/or translational apparatus of the cells would faithfully mimic the response of normal or cancerous breast tissue to an exogenous agent. Such machinery would likely include appropriate surface transduction mechanisms and/or cytosolic factors.

By way of example, and not limitation, examples of the present invention will now be given.

### EXAMPLE 1

Gene expression in cell line of cancer origin.

Cell lines of different origin were analyzed according to the production of the capture nucleotide sequences and of the targets.

The cell lines were:

BT-474 (ESR1+, ERBB2+), HS578T (ESR1−, ERBB2−), MCF-7 (ESR1+, ERBB2−), MDA-MB-231 (ESR1−, ERBB2−), MDA-MB-453 (ESR1−, ERBB2+), T-47D (ESR1+, ERBB2−), as described at ATCC (www.atcc.org). Evsa-T (Borras M. et al., Cancer Lett. 1997 Nov. 25; 120(1):23-30), IBEP-1, IBEP-2, IBEP-3 (Siwek B. et al., Int J Cancer. 1998 May 29;76(5):677-83), KPL-1 (Kurebayashi J. et al., Br J Cancer. 1995 April;71(4):845-53).

1. RNA Extraction:

Frozen tumors, or tumors maintained in RNAlater (Ambion) were crushed in liquid nitrogen, using mortar and pestle. Total RNA was extracted from powdered tumors and cultured breast cancer cells by TriPure (Roche), according to the manufacturer's instructions. Poly(A⁺) RNA (mRNA) was obtained from total RNA using FastTrack columns (InVitrogen). Poly(A⁺) RNA was resuspended in RNAse-free water.

The concentration and purity of RNA was determined by diluting an aliquot of the preparation in TE (10 mM Tris-HCl pH 8, 1 mM EDTA) and measuring (reading) its absorbance (in a spectrophotometer) at 260 nM and 280 nm.

While A260 allows to evaluate the RNA concentration, the A260/A280 ratio gives an indication of RNA purity. For a RNA to be used, its ratio must be comprised between 1.8 and 2.

The overall quality of the RNA preparation was determined by electrophoresis on a denaturing 1% agarose gel (Sambrook et al., eds. (1989) Molecular Cloning—A Laboratory Manual, 2nd ed. Cold Spring Harbor, N.Y.: Cold Spring Harbor Laboratory Press).

2. cDNA Synthesis:

1 μl of poly(A⁺) RNA sample (0.5 μg/μl) was mixed with 2 μl oligo(dT)₁₂₋₁₈ (0.5 μg/μl, Roche), 3.5 μl H₂O, and 3 μl of a solution of 3 different synthetic well-defined poly(A⁺) RNAs. These latter served as internal standards to assist in quantification and estimation of experimental variation introduced during the subsequent steps of analysis. After an incubation of 10 minutes at 70 C and 5 minutes on ice, 9 μl of reaction mix were added. Reaction mix consisted in 4 μl Reverse Transcription Buffer 5×(Gibco BRL), 1 μl RNAsin Ribonuclease Inhibitor (40 U/ml, Promega), and 2 μl of a 10×dNTP mix, made of dATP, dTTP, dGTP (5 mM each, Roche), dCTP (800 μM, Roche), and Biotin-11-dCTP (800 μM, NEN).

After 5 minutes at room temperature, 1.5 μl SuperScript II (200 U/ml, Gibco BRL) was added and incubation was performed at 42 C for 90 minutes. Addition of SuperScript and incubation were repeated once. The mixture was then placed at 70 C for 15 minutes and 1 μl Ribonuclease H (2 U/μl) was added for 20 minutes at 37 C. Finally, a 3-minutes denaturation step was performed at 95 C. The biotinylated cDNA, was kept at −20 C.

3. Hybridization of (Using) Biotinylated cDNA:

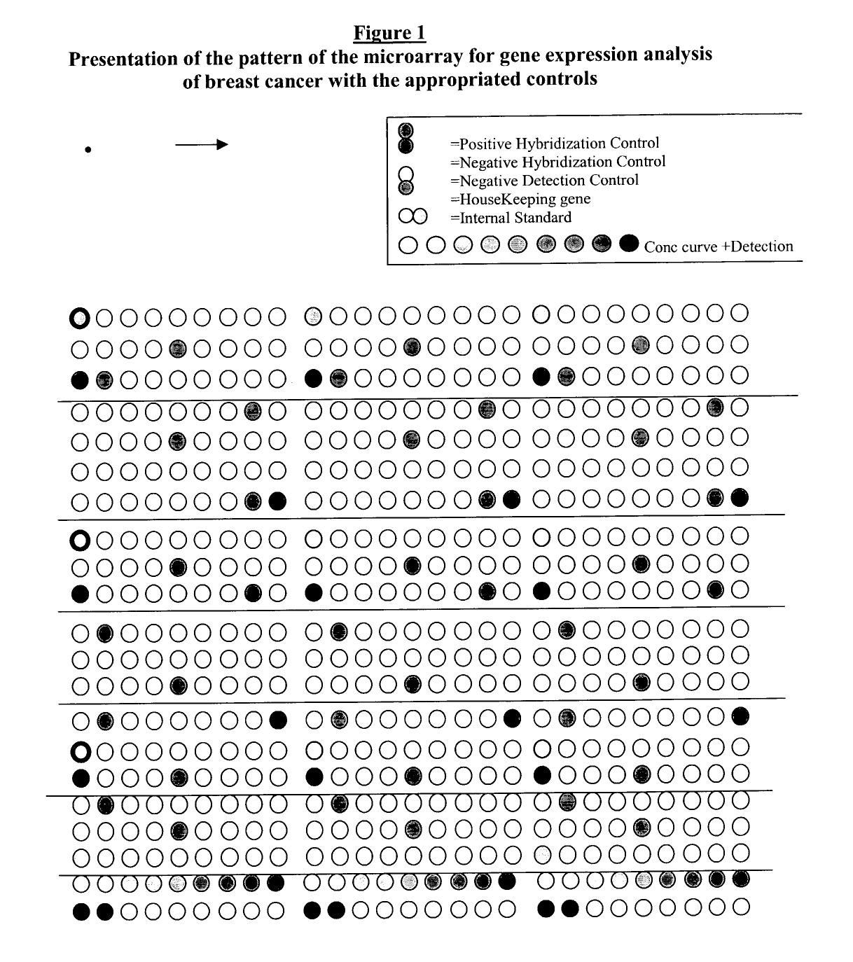

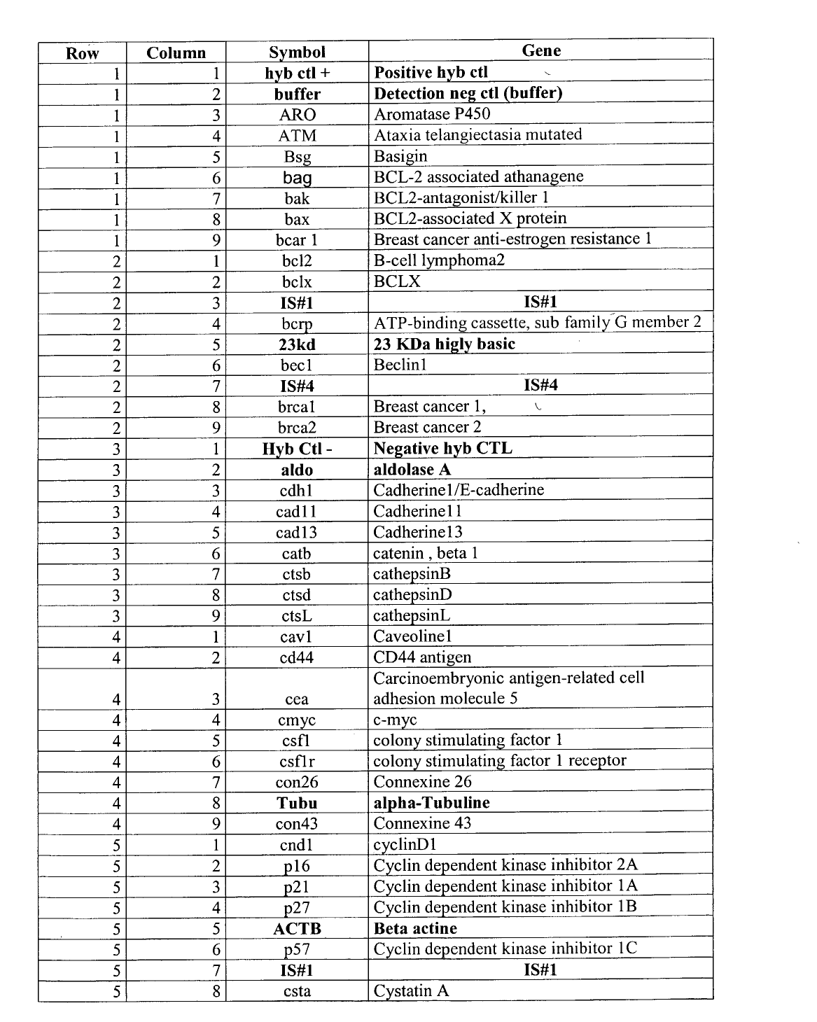

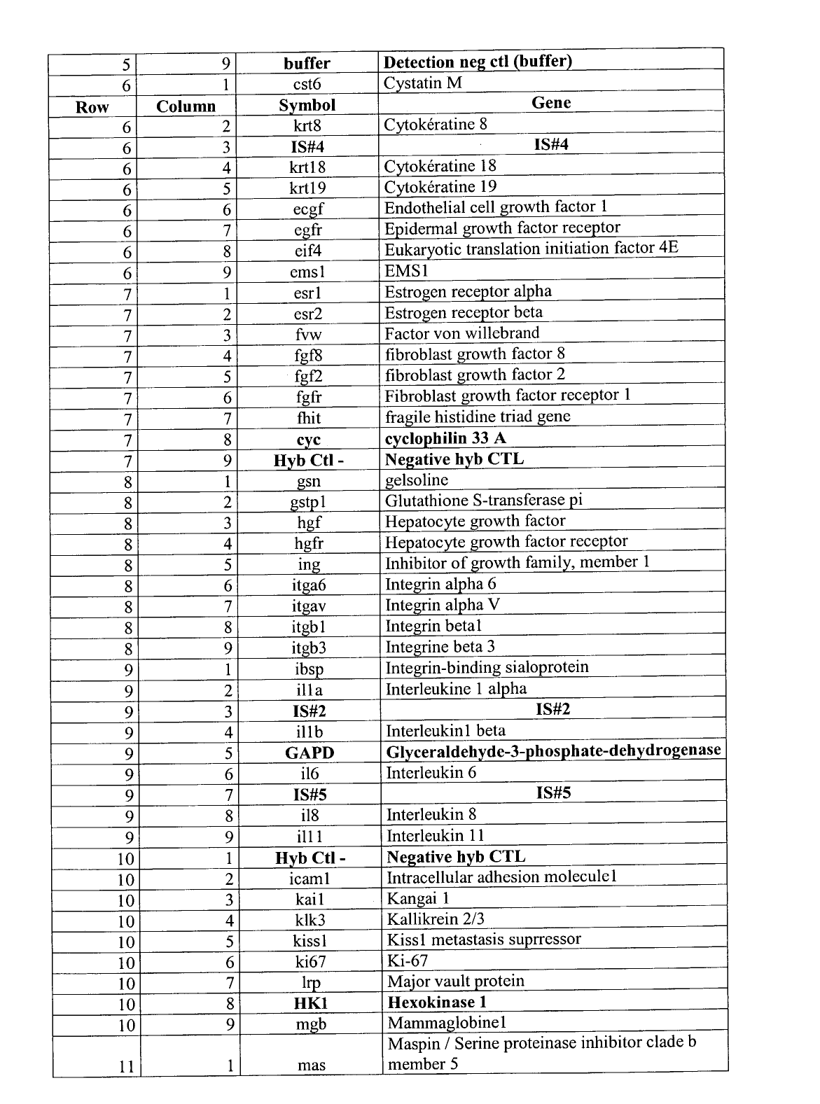

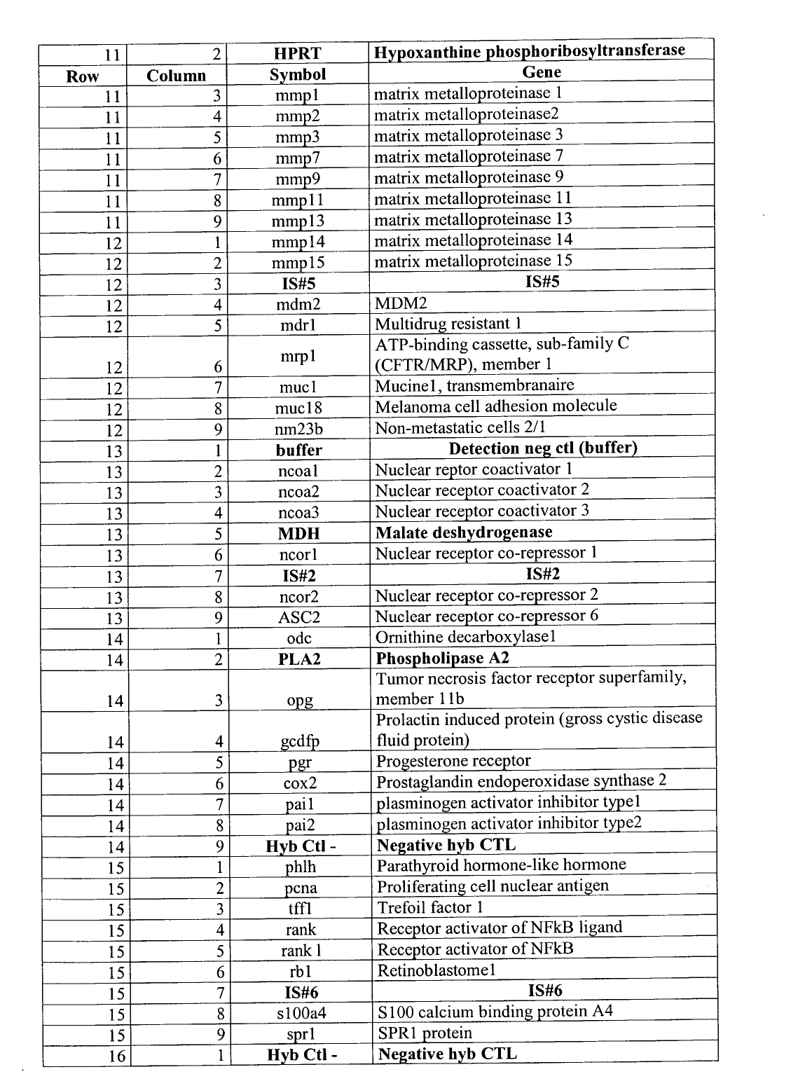

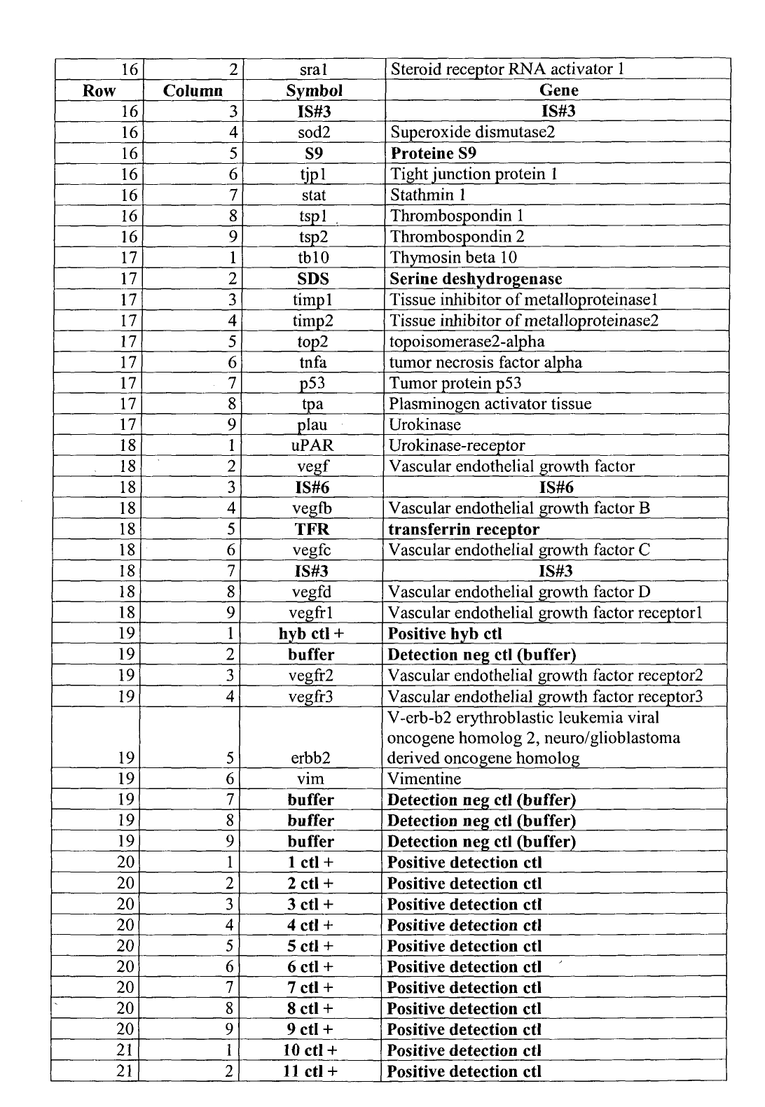

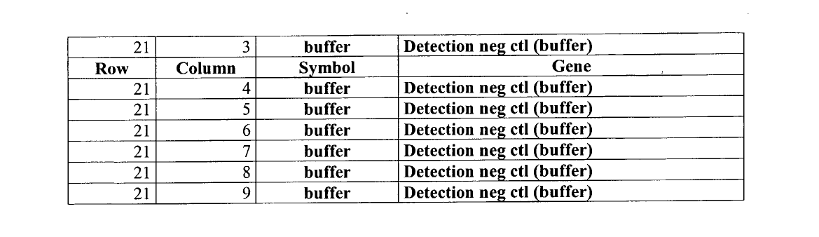

The BreastChips used in this study is composed of 145 genes and several different controls including positive and negative detection control, positive and negative hybridization control, three different internal standards all dispersed at different locations among the genes to be analyzed on the micro-array (FIG. 1). In this example each spots was covered with a capture probe being a polynucleotide species which allow the specific binding of one target polynucleotide corresponding to a specific gene listed in table 2.

Hybridization chambers were from Biozym (Landgraaf, The Netherlands). Hybridization mixture consisted in biotinylated cDNA (the total amount of labeled cDNA), 6.5 μl HybriBuffer A (Eppendorf, Hambourg, Germany), 26 μl HybriBuffer B (Eppendorf, Hambourg, Germany), 8 μl H₂O, and 2 μl of positive hybridization control.

Hybridization was carried out overnight at 60° C. The micro-arrays were then washed 4 times for 2 min with washing buffer (B 1 0.1×+Tween 0.1%) (Eppendorf, Hamburg, Germany).

The micro-arrays were than incubated for 45 minutes at room temperature with the Cy3-conjugated IgG Anti biotin (Jackson Immuno Research Laboratories, Inc #200-162-096) diluted 1/1000× Conjugate-Cy3 in the blocking reagent and protect from light.

The micro-arrays were washed again 4 times for 2 minutes with washing buffer (B1 0.1×+Tween 0.1%) and 2 times for 2 minutes with distilled water before being dried under a flux of N₂.

4. Scanning and Data Analysis:

The hybridized micro-arrays were scanned using a laser confocal scanner “ScanArray” (Packard, USA) at a resolution of 10 μm. To maximize the dynamic range of the assay the same arrays were scanned at different photomultiplier tube (PMT) settings. After image acquisition, the scanned 16-bit images were imported to the software, ‘ImaGene4.0’ (BioDiscovery, Los Angeles, Calif., USA), which was used to quantify the signal intensities. Data mining and determination of significantly expressed gene in the test compared to the reference arrays was performed according to the method described by Delongueville et al (Biochem Pharmacol. 2002 Jul. 1;64(1):137-49). Briefly, the spots intensities were first corrected for the local background and than the ration between the test and the reference arrays were calculated. To account variation in the different experimental steps, the data obtained from different hybridizations were normalized in two ways. First the values are corrected using a factor calculated from the intensity ratios of the internal standard reference and the test sample. The presence of 3 internal standard probes at different locations on the micro-array allows measurement of a local background and evaluation of the micro-array homogeneity, which is going to be considered in the normalization (Schuchhardt et al., Nucleic Acids Res. 28 (2000), E47). However, the internal standard control does not account for the quality of the mRNA samples, therefore a second step of normalization was performed based on the expression levels of housekeeping genes. This process involves calculating the average intensity for a set of housekeeping genes, the expression of which is not expected to vary significantly. The variance of the normalized set of housekeeping genes is used to generate an estimate of expected variance, leading to a predicted confidence interval for testing the significance of the ratios obtained (Chen et al, J. Biomed. Optics 1997, 2, 364-74). Ratios outside the 95% confidence interval were determined to be significantly changed by the treatment.

In this experiment, the ratios of gene expressed in each cell type was compared with the average gene expression obtained from a mixture of mRNA isolated from the 11 cell lines.

### EXAMPLE 2

Gene expression in breast cancer.

The RNA extraction, cDNA preparation, hybridization, and quantification of the array were performed as described in the example 1 from tumors tissues obtained from chirurgical intervention.

The tumor cDNA was analyzed on an array present on a slide with the presence of another array. This second array serves as reference and was hybridized from cDNA obtained as a mixture of the RNA from the 12 cell lines used in the example 1. The identification and quantification of the genes differentially expressed in tumors were obtained from the comparison of the hybridization yield of the two arrays.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A micro-array for the diagnosis and prognosis of human breast cancer comprising:
a solid support; and
a plurality of capture probes, present on the solid support in the form of an array, which are selected from the group consisting of polynucleotides, polypeptides and fragments thereof, derived from
(a) at least one gene representing at least 4 out of 6 phenotypes, selected from the group consisting of luminal/epithelial, basal/myoepithelial, mesenchymal, ErbB2, hormonal phenotypes and hereditary susceptibility to breast cancer; and
(b) at least 10 genes associated with at least 3 cellular functions.

2. The micro-array according to claim 1, which comprises capture probes for the detection of not more than 3000 different genes.

3. The micro-array according to claim 1, wherein each gene is quantified on an array by spots with single nucleotide species.

4. The micro-array according to claim 1, wherein each gene is quantified on an array by spots with attached or adsorbed antibodies specific of the proteins derived from the expressed genes.

5. The micro-array according to claim 1, wherein one spot is sufficient for the identification and quantification of one gene or gene product.

6. The micro-array according to claim 1, wherein a spot intensity is read in fluorescence or colorimetry.

7. The micro-array according to claim 1, wherein the quantification of the genes is performed by comparison with internal standards introduced into the retro-transcription of mRNA.

8. The micro-array according to claim 1, wherein the solid support is selected from the group consisting of glass, plastic, filters, metals and electronic chips.

9. A method for the diagnosis and prognosis of breast cancer, comprising the steps of:
providing a micro-array comprising a solid support and a plurality of capture probes, present on the solid support in the form of an array, which are selected from the group consisting of polynucleotides, polypeptides and fragments thereof, and quantifying the different expression of at least 4 of the 6 cancer phenotypes and at least 10 other genes associated with at least 3 cellular functions and/or house keeping functions.

10. The method according to claim 9, comprising the step of performing the method on cDNA obtained by retro-transcription from less than 20 μg of total RNA.

11. The method according to claim 9, comprising the step of performing the method on cDNA obtained by retro-transcription from less than 10 μg of total RNA.

12. The method according to claim 9, including the step of obtaining by retro-transcription from less than 1 μg of mRNA.

13. The method according to claim 9, wherein the differentially expressed genes are quantified and/or identified by reference with a reference tissue or reference cell.

14. The method according to claim 9, wherein the results on the breast cancer and to a reference material is obtained on micro-arrays present on the same support.

15. The method according to claim 9, wherein the density of the capture nucleotide sequences bound to the surface of the solid support is superior to 3 fmoles per cm of solid support surface.

16. The method according to claim 9, wherein the insoluble solid support is selected from the group consisting of glasses, electronic devices, silicon supports, plastic supports, compact discs, filters, gel layers, metallic supports and a mixture thereof.

17. The method according to claim 9, wherein the nucleotide sequences to be detected and to be quantified are RNA sequences obtained by retro-transcription of the 3′ or 5′ end of the transcript by using consensus primer and possibly a stopper sequence

18. The method according to claim 9, wherein the copied or amplified sequences are detected without previous cutting of original sequences into smaller portions

19. A diagnostic kit, comprising:
an array for a solid support;
a plurality of captures probes, present on the solid support in the form of an array, which are selected from the group consisting of polynucleotides, polypeptides and fragments thereof, derived from at least 4 of the 6 cancer phenotypes selected from the group consisting of luminal/epithelial, basal/myoepithelial, mesenchymal, ErbB2, hormonal phenotypes and hereditary susceptibility to breast cancer and at least 10 other genes associated with at least 3 cellular functions.

20. The kit according to claim 19, wherein the solid support is selected from the group consisting of glass, silicon, plastic, filters, gel layers, metal and a mixture thereof.

21. A micro-array for the detection of cancer comprising:
a solid support; and
a plurality of captures probes, present on the solid support in the form of an array, which are selected from the group consisting of polynucleotides, polypeptides and fragments thereof, derived from
(a) at least one gene representing at least 4 out of 6 phenotypes, selected from the group consisting of luminal/epithelial, basal/myoepithelial, mesenchymal, ErbB2, hormonal phenotypes and hereditary susceptibility to breast cancer; and
(b) at least 10 genes associated with at least 3 cellular functions.

22. A micro-array for the determination of the progress of cancer comprising:
a solid support; and
a plurality of captures probes, present on the solid support in the form of an array, which are selected from the group consisting of polynucleotides, polypeptides and fragments thereof, derived from
(a) at least one gene representing at least 4 out of 6 phenotypes, selected from the group consisting of luminal/epithelial, basal/myoepithelial, mesenchymal, ErbB2, hormonal phenotypes and hereditary susceptibility to breast cancer; and
(b) at least 10 genes associated with at least 3 cellular functions.

23. The micro-array of claim 21, wherein the cancer is ductal carcinogenesis.

24. The micro-array of claim 22, wherein the cancer is ductal carcinogenesis.

25. A micro-array for the detection of cancerogenous agents and/or for the detection of cytostatic or antiproliferative agents comprising:
a solid support; and
a plurality of captures probes, present on the solid support in the form of an array, which are selected from the group consisting of polynucleotides, polypeptides and fragments thereof, derived from
(a) at least one gene representing at least 4 out of 6 phenotypes, selected from the group consisting of luminal/epithelial, basal/myoepithelial, mesenchymal, ErbB2, hormonal phenotypes and hereditary susceptibility to breast cancer; and
(b) at least 10 genes associated with at least 3 cellular functions.

26. A method for the diagnosis and prognosis of breast cancer, comprising the steps of:
providing a micro-array comprising a solid support containing a plurality of capture probes which are selected for the detection of a group comprising polynucleotides or polypeptides derived from at least 4 expressed genes or its complement representing the hereditary phenotype and at least 4 genes associated with other cellular functions and/or house keeping genes.
